(19) United States
(12) Patent Application Publication
(10) Pub. No.: US 2025/0357030 A1
(43) Pub. Date: Nov. 20, 2025

(54) SYSTEMS AND METHODS USING ROTATING MAGNETIC ROLLERS

(71) Applicant: 3M INNOVATIVE PROPERTIES COMPANY, St. Paul, MN (US)

(72) Inventors: Ronald D. Jesme, Plymouth, MN (US); Joseph B. Eckel, Vadnais Heights, MN (US); Aaron K. Nienaber, Lake Elmo, MN (US); Christian Weinmann, Alsdorf (DE); Nitsan Ben-Gal Nguyen, Apple Valley, MN (US)

(21) Appl. No.: 18/867,709

(22) PCT Filed: May 16, 2023
(86) PCT No.: PCT/IB2023/055043

§ 371 Date: Nov. 20, 2024

Related U.S. Application Data
(60) Provisional application No. 63/365,411, filed on May 26, 2022.

(51) Int. Cl. H01F 7/02

(52) U.S. Cl. CPC H01F 7/0205

(57) ABSTRACT

Mechanical systems including a pair of rotatable magnetic rollers are provided. Each roller includes a set of magnets mounted on a rotating shaft. Each magnet in the sets is diametrically magnetized. Methods of reducing or eliminating a torque to rotate the magnetic rollers are provided.

### BACKGROUND

Rotating magnets can be used to align particles to enable the production of advanced abrasive, magnetic, electrical thermal, and optical articles. For example, PCT Patent Publication No. WO 2018/136268 (to Jesme et al.) describes methods of making an abrasive article by varying a magnetic field relative to magnetizable abrasive particles on a surface to impart a non-random orientation and/or alignment to the magnetizable abrasive particles.

### SUMMARY

In one aspect, the present disclosure describes a mechanical system including a first magnetic roller including a first set of magnets mounted on a first rotating shaft extending along a first rotation axis, and a second magnetic roller including a second set of magnets mounted on a second rotating shaft along a second rotation axis substantially parallel to the first rotation axis. The first and second magnetic rollers are positioned with a gap therebetween. Each magnet in the first and second sets is diametrically magnetized with a magnetic orientation substantially perpendicular to the first or second rotation axis.

In another aspect, the present disclosure describes a method including positioning a first magnetic roller extending along a first rotation axis and a second magnetic roller extending along a second rotation axis substantially parallel to the first rotation axis. The first and second magnetic roller each include a first or second set of magnets mounted on a first or second rotating shaft. Each magnet in the first and second sets is diametrically magnetized with a magnetic orientation substantially perpendicular to the first or second rotation axis. The method further includes rotating the first and second magnetic rollers with a torque.

Various unexpected results and advantages are obtained in exemplary embodiments of the disclosure. One such advantage of exemplary embodiments of the present disclosure is that the torque required to initiate and complete a rotation of a pair of magnetic rollers is minimized, which also reduces power consumption, motor size, motor cost, mechanical vibration, and variability in rotation speed over the course of rotation.

Various aspects and advantages of exemplary embodiments of the disclosure have been summarized. The above Summary is not intended to describe each illustrated embodiment or every implementation of the present certain exemplary embodiments of the present disclosure. The Drawings and the Detailed Description that follow more particularly exemplify certain preferred embodiments using the principles disclosed herein.

### BRIEF DESCRIPTION OF THE DRAWINGS

The disclosure may be more completely understood in consideration of the following detailed description of various embodiments of the disclosure in connection with the accompanying figures, in which:

FIG. 1 is a side perspective view of a magnetic roller system, according to one embodiment.

FIG. 2A is a side perspective view of a pair of magnetic rollers, according to one embodiment.

FIG. 2B is a side perspective view of a pair of magnetic rollers, according to another embodiment.

FIG. 2C is a map of magnetic flux for the pair of magnetic rollers of FIG. 2B

FIG. 3A is a schematic view of a magnet, according to one embodiment.

FIG. 3B is a schematic view of a set of magnets, according to one embodiment.

FIG. 4A is a schematic view of a pair of magnetic rollers in a first position and its associated magnetic field, according to one embodiment.

FIG. 4B is a schematic view of the pair of magnetic rollers of FIG. 4A in a second position and its associated magnetic field.

FIG. 5 is a schematic view of a pair of magnetic rollers, according to one embodiment.

FIG. 6A is a schematic diagram of a magnetic field of a pair of magnetic rollers in a first position, according to another embodiment.

FIG. 6B is a schematic diagram of a magnetic field of the pair of magnetic rollers of FIG. 6A in a second position.

FIG. 7 is a schematic view of a pair of magnetic rollers, according to one embodiment.

FIG. 8 is a schematic view of a pair of magnetic rollers, according to another embodiment.

FIG. 9 is plots of magnetic torque for the pair of magnetic rollers of FIG. 7.

FIG. 10A is a schematic diagram of a mechanical system coupled with a pair of magnetic rollers at a first state, according to one embodiment.

FIG. 10B is a schematic diagram of the mechanical system of FIG. 10A at a second state.

FIG. 10C is a schematic diagram of the mechanical system of FIG. 10A at a third state.

In the drawings, like reference numerals indicate like elements. While the above-identified drawing, which may not be drawn to scale, sets forth various embodiments of the present disclosure, other embodiments are also contemplated, as noted in the Detailed Description. In all cases, this disclosure describes the presently disclosed disclosure by way of representation of exemplary embodiments and not by express limitations. It should be understood that numerous other modifications and embodiments can be devised by those skilled in the art, which fall within the scope and spirit of this disclosure.

### DETAILED DESCRIPTION

There is a desire to use a stronger magnetic field, for example, in a production process that can provide a range of advantages including, for example, the ability to manipulate less magnetic and/or lower-cost particles on a web, the ability to better align particles, the ability to run at faster line speeds, etc. One way to provide a greater magnetic field strength is to add a second counterrotating magnet above the web line to form a pair of magnets with the web passing therebetween. The pair of magnets may have a strong tendency to remain magnetically aligned, and the motors required to spin up the magnets may need to be unusually large to develop the torque needed during startup to overcome the strong magnetic attraction. This disclosure provides, in some embodiments, various means of reducing or eliminating such a torque requirement, enabling the use of much smaller (and/or less expensive) motors and motor controllers to rotate the magnets, allowing the equipment to fit within the space available of many pilot and production web lines.

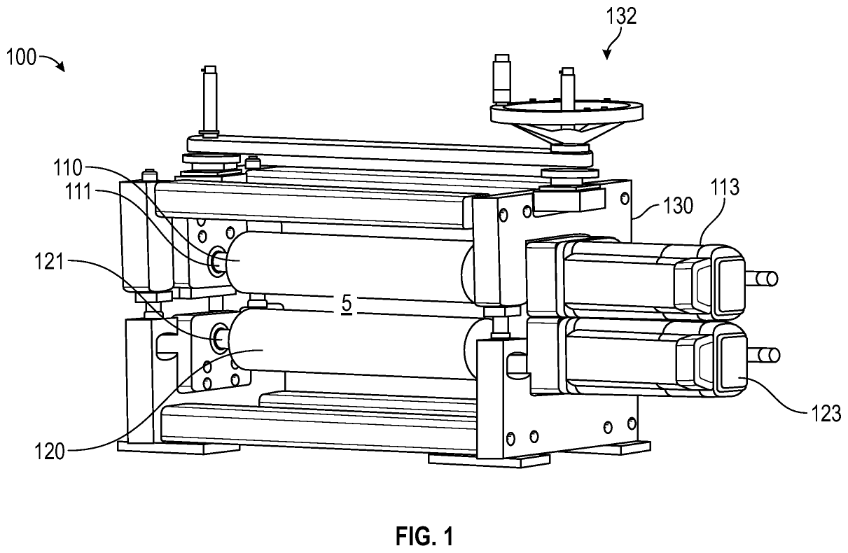

FIG. 1 is a side perspective view of a motorized mechanical system 100 of rotating magnets, according to one embodiment. The system 100 includes a first magnetic roller 110 formed by mounting a first set of magnets on a first rotating shaft 111, and a second magnetic roller 120 formed by mounting a second set of magnets on a second rotating shaft 121. The magnetic rollers 110, 120 each are mounted on a mounting and positioning mechanism 130 such that the first shaft 111 extends along a first rotation axis and the second shaft 121 extends along a second rotation axis substantially parallel to the first rotation axis. The mounting and positioning mechanism 130 further include cranks and/or wheels 132 used to adjust the gap 5 between the magnetic rollers 110, 120. The system 100 further includes a first motor 113 mechanically connected to the first rotating shaft 111 to rotate the first magnetic roller 110, and a second motor 123 mechanically connected to the second rotating shaft 121 to rotate the second magnetic roller 120.

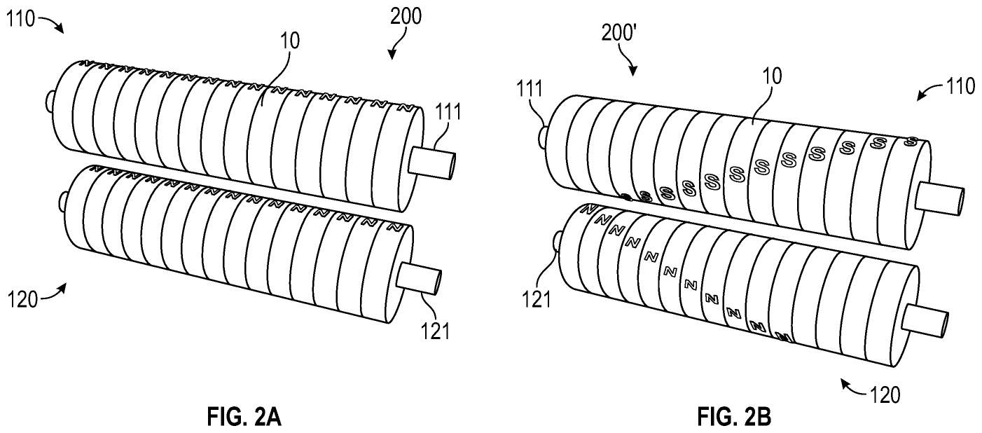

The first and second magnetic rollers 110, 120 each include a set of magnets. FIG. 2A is a side perspective view of a magnetic assembly 200 including a pair of magnetic rollers 110, 120. Each roller includes an array of disc-shaped magnets 10 mounted on the rotating shafts 111, 121, according to one embodiment. FIG. 2B is a side perspective view of a magnetic assembly 200′ including a pair of disc-shaped magnetic rollers 110, 120 each including magnets 10 mounted on the rotating shafts 111, 121, according to another embodiment.

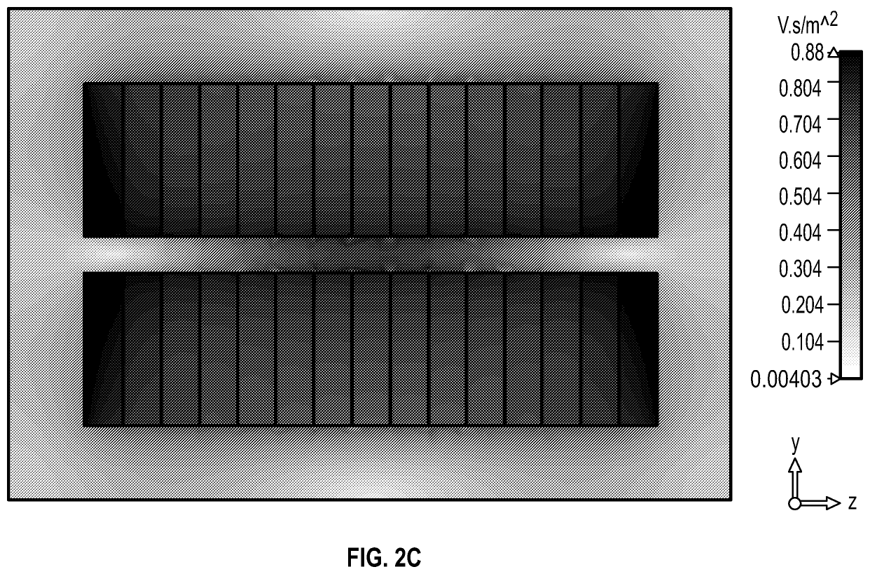

An exemplary magnet 10 is illustrated in FIG. 3A, according to some embodiments. The magnet 10 is a diametrically magnetized cylinder or disc that includes two poles N and S that are each shaped as hemispheres and are disposed to either side of the axis of rotation AR. The magnetic orientation of a magnet is shown by an arrow pointing from the S pole to the N pole. The magnet 10 has a width w in the range, for example, from 0.5 cm to 7.0 cm, and a diameter d in the range, for example, from 0.5 cm to 13 cm. It is to be understood that the sizes of a magnet may be related to practical magnet construction limitations.

FIG. 3B is a schematic view of a magnet assembly 30, according to one embodiment. The magnet assembly 30 includes a first magnet 10a and a second magnet 10b each being a diametrically magnetized cylinder or disc. The orientations of first and second magnets 10a, 10b are rotated with respect to each other about the rotating axis A such that the pole N of the first magnet 10a does not align directly with the pole N of the second magnet 10b. Instead, the first and second magnets 10a, 10b are angularly displaced with respect to the axis A with an angle a between the respective orientations. The first and second magnets can be mounted on a rotating shaft (e.g., the shafts 111, 121 of FIG. 2A-B) with the respective axes being aligned along the rotation axis A. The respective orientations of the first and second magnets 10a, 10b are angularly displaced or shifted with respect to the axis A. It is to be understood that in some embodiments, the magnet assembly 30 may be a composite assembly including a first portion as the first magnet and a second portion as the second magnet, and the first and second portions are integrated as a one-piece structure. It is also to be understood that the magnet assembly 30 may include two or more integrated portions/magnets assembled along the axis A.

Referring again to FIGS. 2A and 2B, the first and second magnetic rollers 110, 120 each include a number N of the magnets 10. The number N may be in the range of, for example, from 4 to 50. It is to be understood that the number N may depend on the desired applications. The axes of the magnets 10 in each magnetic roller 110, 120 are aligned along the respective rotation axes 111, 121. The orientations of magnetic poles for each cylinder 10 is substantially perpendicular to the respective rotation axes 111, 121. With the magnets 10 assembled along the rotating axis, each magnetic roller 110, 120 has a width W=Nw, where N is the number of magnets in the magnetic roller, and w is the width of each magnet 10. It is to be understood that a thin layer of glue or other suitable material may be used to assemble the magnets. The roller width W may be substantially the same as or comparable to the width of a web to pass between the pair of magnetic rollers 110, 120.

In one application, the mechanical systems described herein can be used to manipulate magnetic or magnetizable particles on a substrate surface such as a web. The magnetic or magnetizable particles supported by the substrate surface can pass between the pair of magnetic rollers, where the magnetic field from the rotating rollers can manipulate the particles such as, for example, assemble the particles into a desired structure, impart a non-random orientation and/or alignment to the magnetic or magnetizable particles relative to the substrate surface. In some embodiments, the particles can be added, for example, via a drop coater, to the substrate while it is within the magnetic field of the magnetic rollers.

Suitable magnetic or magnetizable particles may include particles formed from any of the magnetizable materials described elsewhere, optionally coated with another material, and particles formed from a non-magnetizable material and coated with a magnetizable material. For example, suitable magnetizable particles include nickel-coated graphite flakes, nickel-coated glass spheres, and nickel-coated plastic particles (e.g., nickel coated polymethyl methacrylate (PMMA) particles).

In the embodiment 200 depicted in FIG. 2A, the magnets 10 in each roller 110, 120 have their N and S poles aligned. When motors are not energized to rotate the rollers, the N poles of the magnets in one magnetic roller are magnetically attracted to the S poles of the magnets in the other magnetic roller. It was found in this disclosure that the smaller the gap g between the pair of magnetic rollers, the larger the torque being required to rotate the magnetic rollers to initiate rotation. In some embodiments, the gap g between the pair of magnetic rollers can be adjusted to greater than a critical value to initiate the rotation. In some embodiments, the gap may be in the range, for example, from about 0.005 cm to about 100 cm, from about 0.01 cm to about 50 cm, or from about 0.05 cm to about 30 cm. For a given configuration of the pair of rollers (i.e., the numbers, the sizes, and the magnetic properties of the magnets, the gap between the pair of rollers, etc.), the torque T₀ required to rotate the magnetic rollers can be experimentally determined.

The present disclosure provides various embodiments to minimize the maximum torque needed to initiate and/or complete a rotation of a pair of magnetic rollers. It is to be understood that at some angular positions of a full rotation, the torque T₀ required may be higher than at other angular positions. The various embodiments can minimize the highest (or maximum) torque needed to complete a full rotation. The above torque T₀ for the configuration in FIG. 2A can be used as a reference torque when comparing to the reduced or minimized torque.

In the embodiment 200′ depicted in FIG. 2B, the adjacent magnets 10 in each magnetic roller 110, 120 have their N and S poles angularly displaced or shifted substantially equally with an angle of 180°/N, where N is the number of magnets in the respective magnetic rollers 110, 120. For example, the exemplary rollers in FIG. 2B each include 15 magnetic units and the angle is about 180°/15=12 degrees. That is, the pole of each magnet is angularly displaced equally by 12 degrees, with the intent of one set of poles pulling together as another set of poles are being pulled apart, to eliminate or greatly minimize the net torque needed to rotate the magnetically coupled rollers. With such angular shift or displacement, the torque T₁ required to complete a rotation of the magnetic rollers can be reduced as compared to the torque T₀ required to complete a rotation of the magnetic rollers in FIG. 2A. In some embodiments, the torque T₁ may have a value in the range, for example, from about 50% to about 0.5%, from about 30% to about 1%, or from about 30% to about 5% of that of the torque T₀.

In the present disclosure, simulation tools have been used to obtain information regarding the shape and the distribution of magnetic fields for various configurations of magnetic-roller pairs. In some cases, the software CST Studio from Dassault Systemes was used. A full three-dimensional computer-aided design (3D CAD) representation of the magnets was used and calculated by a Magnetostatic Solver. According to one example, the 3D Model consists of two rows (rollers) of each 15 magnetic discs as shown in FIG. 2B with a variable distance. Each disc can be pre-set with a rotation angle offset against each other disc. All discs of a roller can be furthermore rotated with a total angle value. Thus, any angle offset between the neighboring disks and any rotation angle of each roller can be simulated and visualized. As the distance between the rollers is also parametrized, sweeps of the influence of the distance between the rollers on the magnetic field shapes can be visualized. The results of the simulation can be shown as 2D or 3D representations of the magnetic field vectors generated by the actual setup and rotation of the magnets. The magnetic field can be either shown as an absolute value to get the overall field magnitude, or only in x, y, or z direction in a (x, y, z) cartesian system.

FIG. 2C illustrates a map of magnetic flux for the configuration 200′ of FIG. 2B, according to CST modeling and simulation results. In the setup as shown in FIG. 2C, the y-component of the magnetic field vector is a measure for the force between the two rollers which should be constant for all roller-rotation angles. Z and x components are undesired and should be designed to be minimal as part of the design optimization process. The magnetic field of each magnet is marked with different grayscales, and the darker the grayscale, the stronger the magnetic field.

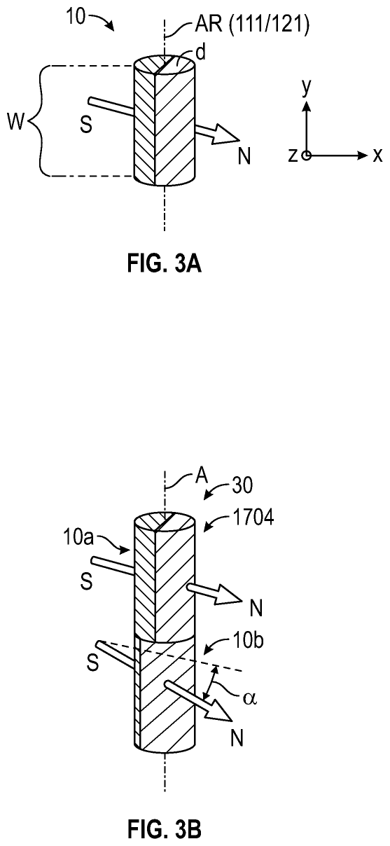

The modeling and simulation results reveal that the magnetic flux crowds toward the ends of the magnet roller, resulting in an unequal flux density along the rotation axial direction. FIG. 4A further illustrates the net system torque for the configuration 200′ of FIG. 2B. It is to be noted that arrows are used to represent a simplified example with seven magnets (e.g., magnetic discs in this example) per magnetic roller. The magnetic orientations of the adjacent magnets in each roller are angularly displaced, substantially equally by an angle a. As shown in FIG. 4A, the end magnets have a stronger field. FIG. 4B illustrates that when the magnetic rollers are rotated 45 degrees as indicated by the arrows, the stronger magnetic poles at the end of the roller will tend to realign, overcoming the attracting force of the weaker poles in the middle of the roller, causing the magnetic assembly to revert to the orientation shown in FIG. 4A.

It was found in this disclosure that when the flux is more equally distributed along the rotation axis than that in the map of FIG. 2C, the torque required to separate the poles at the ends of the cylinder can be canceled out by the torque pulling the poles together at the center of the magnetic roller. This disclosure provides some embodiments of magnetic assembly having further reduced mechanical drive requirements.

In various embodiments, mechanical systems including a pair of magnetic rollers are provided with a reduced torque (as compared to the reference torque T₀) to complete a rotation of the rollers. The reference torque T₀ refers to a torque to initiate and complete a rotation of the rollers 110, 120 in the system 200 shown in FIG. 2A, where the magnetic orientations of the magnets in each set are aligned to be substantially parallel. Given the same number of the same magnets (e.g., the magnet 10 in FIG. 3A) used in a system and the same gap between the pair of rollers, various configurations are provided to reduce the torque to no greater than 50%, no greater than 30%, no greater than 20%, or optionally, no greater than 10% of the reference torque. In some embodiments, the torque may be reduced to substantially zero.

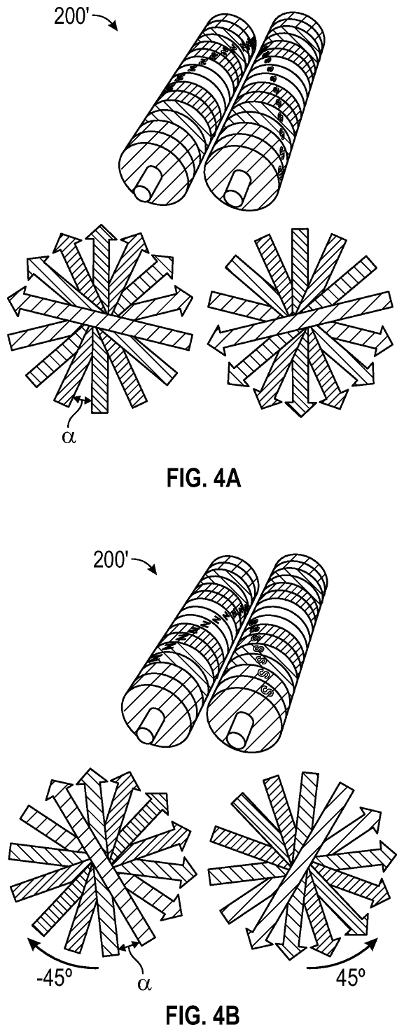

FIG. 5 illustrates a schematic view of a mechanical assembly 500, according to one embodiment. The assembly 500 includes first and second magnetic rollers 110, 120 each including a set of magnets 10 which are arranged in the same configuration as that of FIG. 2B. The first and second magnetic rollers 110, 120 each further includes a set of compensation magnets 12 located adjacent to an end of the set of magnets 10.

The two sets of compensation magnets 12 can be oriented to repel one another at the angular rotation at which the magnets 10 of the magnet rollers 110, 120 tend to attract one another. For example, the north poles of one set of compensation magnets can repel the North poles of the other set of compensation magnets. Each set of compensation magnets 12 may include a suitable number n of magnets 10 having their respective poles aligned along the rotation shafts 111, 121. The poles of the compensation magnets 12 can be aligned with the adjacent end magnet 10 in the respective rollers 110, 120. The number n can be experimentally determined. In some embodiments, the number ratio n/N may be in the range, for example, from 0.01 to 0.5, from 0.01 to 0.3, or from 0.01 to 0.2, where N is the number of magnets 10, and n is the number of compensation magnets 12 for each magnetic roller. For example, it was experimentally determined that 2 magnets in each compensating assembly did not completely offset the torque produced by the ends of the sets of magnets 10, and 3 magnets in each compensating assembly over-compensated and the system tended to come to rest with the North pole of one compensation set aligned with the South pole of the other compensation set.

It was found in this disclosure that a slight offset of the two sets of compensation magnets with respect to each other along the length of the shaft 111, 121 can reduce the over-compensation effect of the compensation magnets 12. For example, in the embodiment depicted in FIG. 5, the two sets of three compensation magnets for the respective magnetic rollers 110, 120 can be positioned offset relative to each other to just the amount needed to substantially offset the residual torque of the system as discussed above. It is to be understood that the gaps g1 and g2 between the set of magnets 10 and the set of compensation magnets 12 for the rollers 110, 112 can be adjusted to achieve the desired compensation effects.

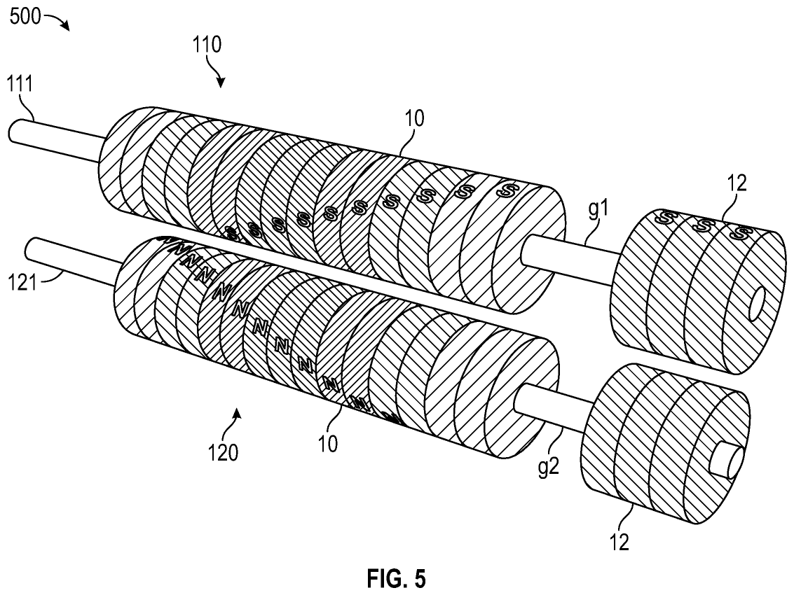

Referring again to FIG. 4A, the orientations of the adjacent magnetic units 10 of the same magnetic roller are angularly displaced or shifted substantially equally with an angle of a=180°/N, where N is the number of magnets in the respective magnetic rollers 110, 120. It is found in this disclosure that when the orientations of the adjacent magnetic units have an unequal angular spacing, it may result in a torque that is near enough to zero for a range of desirable gaps between the magnetic rollers to be practically implemented. FIG. 6A further illustrates the net system torque for a configuration 600 modified from the configuration 200′ of FIG. 2B, where the displacement angles a′ for the configuration 600 are unequal as compared to the displacement angles a in the configuration 200′ of FIG. 4A or 4B are substantially equal. In some embodiments, the end magnets may be more sparsely spaced as compared to the magnets in the middle. It is to be noted that arrows are used to represent a simplified example with seven magnetic units (e.g., discs in this example) per magnetic roller. The magnetic field of each magnet is marked with different grayscales, and the darker the grayscale, the stronger the magnetic field. The magnetic orientations of the adjacent magnets in each roller are angularly displaced by different angles a′. As shown in FIG. 6A, the end magnets that have a stronger field and the strongest magnets are more sparsely spaced angularly as compared to that in FIG. 4A. FIG. 6B illustrates that when the magnetic rollers are rotated 45 degrees as indicated by the arrows, in the position shown, the stronger but more sparsely spaced magnetic poles at the edge can be offset by the weaker but more densely spaced central magnetic poles, which may result in a substantially zero net torque.

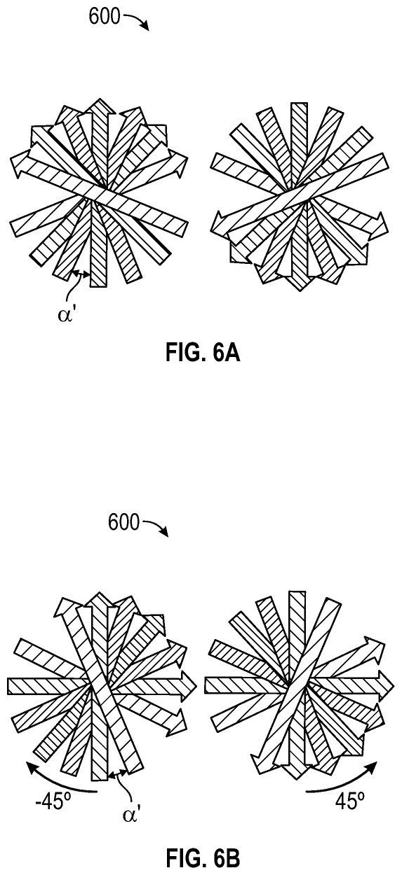

FIG. 7 is a schematic view of a mechanical assembly 700, according to one embodiment. The assembly 700 includes a pair of magnetic rollers 110, 120 magnetically coupled with each other. The magnetic roller 110 includes a first set of magnets 710a and a second set of magnets 720a mounted on a first rotating shaft 111 with a gap therebetween. The magnetic roller 120 includes a first set of magnets 710b and a second set of magnets 720b mounted on a second rotating shaft 121 with a gap therebetween. Each set includes an array of magnets such as the magnet 10 of FIG. 3A, where the orientations of the magnets in each set are aligned along the rotation axis. The set 710a and the set 710b magnetically engages with each other. The set 720a and the set 720b magnetically engages with each other. The set 710a and the set 720a of the first roller 110 have their orientations angularly offset by 90 degrees. The set 710b and the set 720b of the second roller 120 have their orientations angularly offset by 90 degrees.

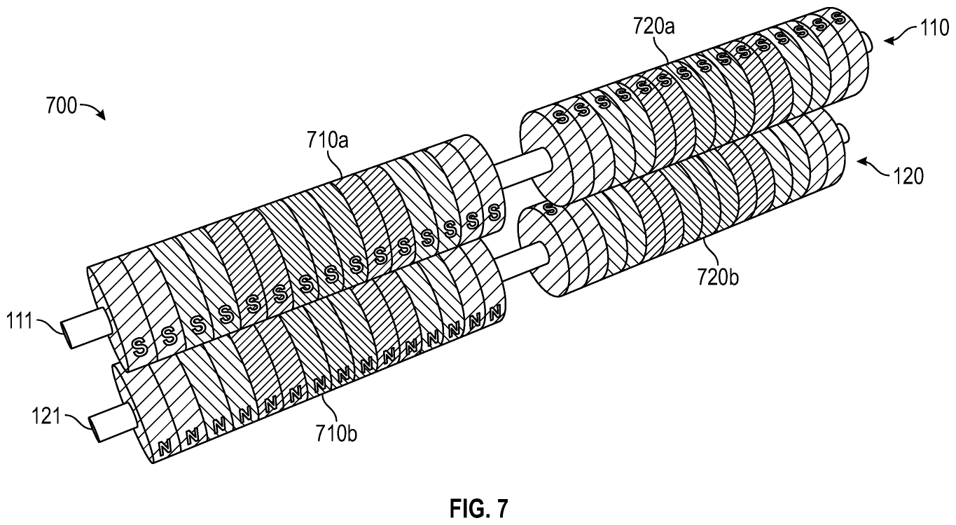

FIG. 8 is a schematic view of a magnetic assembly 800, according to one embodiment. The magnetic assembly 800 includes a pair of magnetic rollers 110, 120 magnetically coupled with each other. The magnetic roller 110 includes a first set of magnets 810a and a second set of magnets 820a mounted on a first rotating shaft 111 with a gap therebetween. The magnetic roller 120 includes a first set of magnets 810b and a second set of magnets 820b mounted on a second rotating shaft 121 with a gap therebetween. Each set includes an array of magnets such as the magnet 10 of FIG. 3A, where the orientations of the magnets in each set are angularly displaced or shifted by an angle a or a′ as discussed above for the configuration in FIG. 4A or 6A. The set 810a and the set 810b magnetically engage with each other. The set 820a and the set 820b magnetically engage with each other. The set 810a and the set 820a of the first roller 110 have their orientations angularly offset by 90 degrees. The set 810b and the set 820b of the second roller 120 have their orientations angularly offset by 90 degrees.

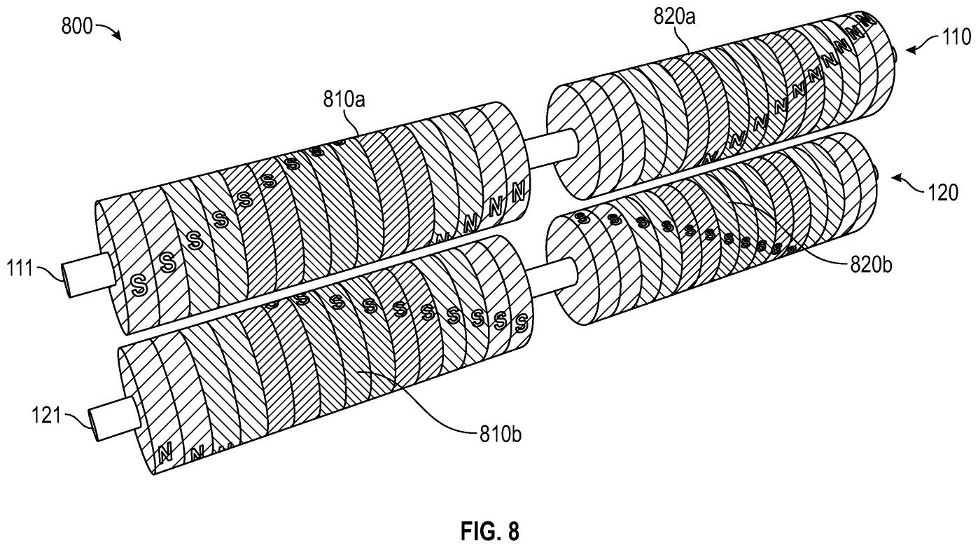

The embodiments shown in FIGS. 7 and 8 provide magnetic roller systems including magnetically straight/twisted mechanically coupled pairs of magnetic sets, which may reduce the required net torque for start rotating the magnetic rollers substantially to zero. FIG. 9 illustrates the torque of the left pair (e.g., 710a and 710b of FIG. 7) and the torque of the right pair (e.g., 720a and 720b of FIG. 7), resulting in a substantially net zero torque.

Mechanical systems described herein show that substantially no (or only very little) energy is needed to maintain the rotation of magnetic rollers and there is no significant energy loss during the rotation of the magnetic rollers, according to some embodiments. For example, as shown in FIG. 9, the torque of the system is approximate sinusoidal, and the net torque over the course of a rotation (or even a ½ rotation) is substantial zero, since the torque is positive for an equal angular displacement as it is negative, with the same magnitude.

Mechanical systems or methods described herein can include various torque reduction mechanisms. One mechanism is for potential energy storage and release. In this approach, the potential energy of the magnetic system can be output and converted to potential energy stored in a mechanical system for part of a rotation. For example, the potential energy can be stored in the form of a compressed spring. The stored mechanical potential energy is converted back to a magnetic potential energy for another part of a rotation. The energy is cycled from one form of energy to the other form, much like the energy of a swinging pendulum oscillates between pure potential energy at the top of the swing to pure kinetic energy at the bottom of the swing. Because the energy of the system is retained (ignoring any system loss due to friction etc.) no substantial additional energy (in the form of torque over some rotational angle) is required to initiate or maintain the spin of the system.

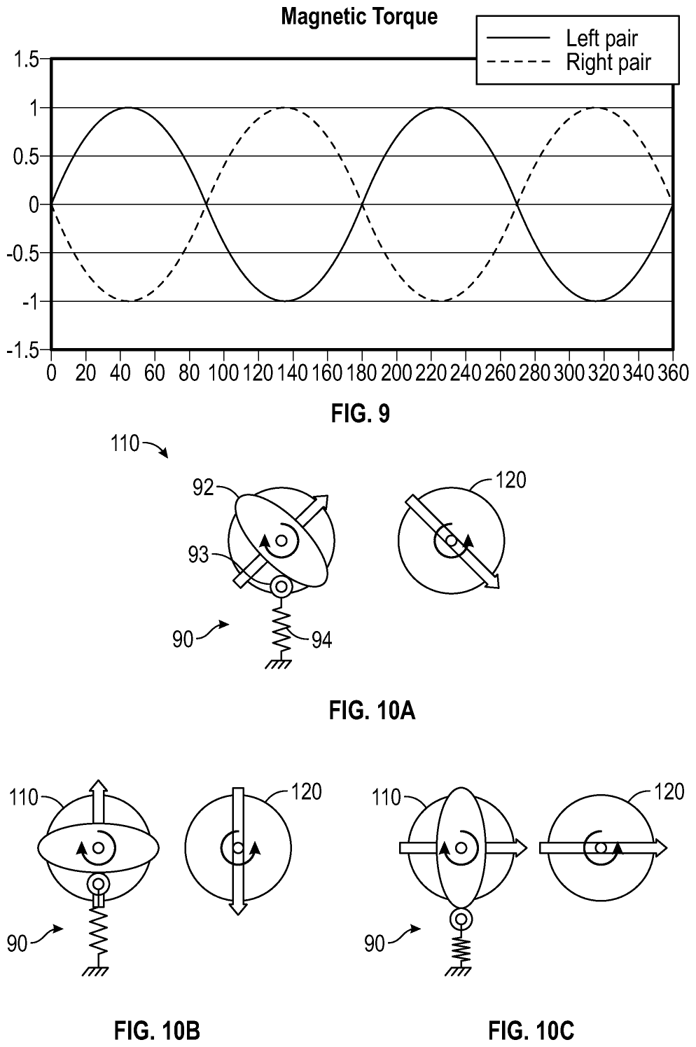

FIGS. 10A-C illustrate a mechanical system 90 functionally connected to the rotating shaft of at least one of the first and second rollers 110, 120 to convert between a magnetic potential energy and a mechanical potential energy of the system. The mechanical system 90 includes a cam 92 fixed to the rotating shaft, and a spring 94 functionally connected to the cam 92 via a cam roller 93. FIG. 10A illustrates the system at a first state with about equal amounts of energy stored in the magnetic potential energy and the mechanical potential energy, in the process of converting more of the magnetic potential energy to the mechanical potential energy. FIG. 10B shows the system in a second state where all the energy is converted to the magnetic potential energy, with the spring 94 decompressed. FIG. 10C shows the system in a third state where substantially all the energy is stored in the spring 94 as the mechanical potential energy.

In some embodiments, the systems or methods described herein can be applied for kinetic energy storage and release. For example, when the system such as that illustrated in FIGS. 10A-C is spinning, the rotational inertia can be used to smooth out the angular velocity of rotation, especially when the amount of rotational kinetic energy is much greater than the amount of energy that is stored as magnetic potential energy. In some embodiments, when it is of interest to have greater stability in the angular velocity over the course of a rotation, then the magnetic torque can be reduced (e.g., by increasing the gap between the pair of magnetic rollers) to make the system to spin faster, and/or the moment of inertia can be increased (e.g., by adding a flywheel to the rotating shaft).

Exemplary embodiments of the present disclosure may take on various modifications and alterations without departing from the spirit and scope of the present disclosure. Accordingly, it is to be understood that the embodiments of the present disclosure are not to be limited to the following described exemplary embodiments, but is to be controlled by the limitations set forth in the claims and any equivalents thereof.

### Listing of Exemplary Embodiments

Exemplary embodiments are listed below. It is to be understood that any one of embodiments 1-12 and 13-23 can be combined.

Embodiment 1 is a mechanical system comprising:
a first magnetic roller including a first set of magnets mounted on a first rotating shaft extending along a first rotation axis; and
a second magnetic roller including a second set of magnets mounted on a second rotating shaft along a second rotation axis substantially parallel to the first rotation axis, the first and second magnetic rollers being positioned with a gap therebetween,
wherein each magnet in the first and second sets is diametrically magnetized with a magnetic orientation substantially perpendicular to the first or second rotation axis.

Embodiment 2 is the system of embodiment 1, wherein the magnetic orientations of the adjacent magnets in the first or second set are angularly displaced.

Embodiment 3 is the system of embodiment 2, wherein the magnets in each set are angularly displaced with an angle of 180°/N, where N is the number of magnets of the first or second set.

Embodiment 4 is the system of embodiment 2, wherein the magnets in each set are angularly displaced with unequal angles.

Embodiment 5 is the system of any one of embodiments 1-4, further comprising a pair of compensation magnets adjacent to the same ends of the first and second sets of magnets, the pair of compensation magnets being positioned to repel one another.

Embodiment 6 is the system of any one of embodiments 1-5, wherein the first set of magnets are arranged as first and second subsets side by side, and the second set of magnets are arranged as first and second subsets side by side, magnetically engaging with the first and second subsets of the first set of magnets, respectively.

Embodiment 7 is the system of embodiment 6, wherein the magnetic orientations of the first and second subsets are angularly offset by about 90 degrees.

Embodiment 8 is the system of any one of embodiments 1-7, further comprising a mechanical system functionally connected to at least one of the first and second rotating shafts to convert between a magnetic potential energy and a mechanical potential energy of the system.

Embodiment 9 is the system of embodiment 8, wherein the mechanical system comprises a cam fixed to at least one of the first and second rotating shafts, and a spring functionally connected to the cam via a cam roller.

Embodiment 10 is the system of any one of embodiments 1-9, further comprising a first motor mechanically connected to the first rotating shaft, and a second motor mechanically connected to the second rotating shaft.

Embodiment 11 is the system of any one of embodiments 1-10, wherein the gap between the first and second rollers are in a range from 0.01 cm to 50 cm.

Embodiment 12 is the system of any one of embodiments 1-11, wherein the first set of magnets and the second set of magnets are positioned such that a torque to rotate the rollers is substantially zero.

Embodiment 13 is a method comprising:
positioning a first magnetic roller extending along a first rotation axis and a second magnetic roller extending along a second rotation axis substantially parallel to the first rotation axis, the first and second magnetic roller each including a first or second set of magnets mounted on a first or second rotating shaft, wherein each magnet in the first and second sets is diametrically magnetized with a magnetic orientation substantially perpendicular to the first or second rotation axis; and
rotating the first and second magnetic rollers with a torque to complete the rotation.

Embodiment 14 is the method of embodiment 13, further comprising reducing the torque to complete the rotation by angularly displacing the magnetic orientation of the adjacent magnets in each set.

Embodiment 15 is the method of embodiment 14, wherein the magnets are angularly displaced with an angle of 180°/N, where N is the number of magnets of the first or second set.

Embodiment 16 is the method of embodiment 14, wherein the magnetic orientations of the magnets in each set are angularly displaced with unequal angles.

Embodiment 17 is the method of any one of embodiments 13-16, further comprising reducing the torque to complete the rotation by disposing a pair of compensation magnets adjacent to the same ends of the first and second sets of magnets.

Embodiment 18 is the method of any one of embodiments 13-17, wherein the first set of magnets are arranged as first and second subsets side by side, and the second set of magnets are arranged as first and second subsets side by side, magnetically engaging with the first and second subsets of the first set of magnets, respectively.

Embodiment 19 is the method of embodiment 18, wherein the magnetic orientations of the first and second subsets are angularly offset by about 90 degrees.

Embodiment 20 is the method of any one of embodiments 13-19, further comprising functionally connecting a mechanical system to at least one of the first and second rotating shafts to convert between a magnetic potential energy and a mechanical potential energy of the apparatus.

Embodiment 21 is the method of embodiment 20, wherein the mechanical system comprises a cam fixed to at least one of the first and second rotating shafts, and a spring functionally connected to the cam via a cam roller.

Embodiment 22 is the method of any one of embodiments 13-21, further comprising adjusting a gap between the first and second magnetic rollers in a range from about 0.01 cm to about 50.0 cm.

Embodiment 23 is the method of any one of embodiments 13-22, further comprising reducing the torque to no greater than 30%, no greater than 20%, or optionally, no greater than 10% of a reference torque, wherein the reference torque refers to a torque to complete the rotation of the first and second magnetic rollers where the magnetic orientations of the magnets in each set are aligned to be substantially parallel.

Reference throughout this specification to “one embodiment,” “certain embodiments,” “one or more embodiments,” or “an embodiment,” whether or not including the term “exemplary” preceding the term “embodiment,” means that a particular feature, structure, material, or characteristic described in connection with the embodiment is included in at least one embodiment of the certain exemplary embodiments of the present disclosure. Thus, the appearances of the phrases such as “in one or more embodiments,” “in certain embodiments,” “in one embodiment,” or “in an embodiment” in various places throughout this specification are not necessarily referring to the same embodiment of the certain exemplary embodiments of the present disclosure. Furthermore, the particular features, structures, materials, or characteristics may be combined in any suitable manner in one or more embodiments. While the specification has described in detail certain exemplary embodiments, it will be appreciated that those skilled in the art, upon attaining an understanding of the foregoing, may readily conceive of alterations to, variations of, and equivalents to these embodiments. Accordingly, it should be understood that this disclosure is not to be unduly limited to the illustrative embodiments set forth hereinabove. Furthermore, various exemplary embodiments have been described. These and other embodiments are within the scope of the following claims.

1. A mechanical system comprising:
a first magnetic roller including a first set of magnets mounted on a first rotating shaft extending along a first rotation axis; and
a second magnetic roller including a second set of magnets mounted on a second rotating shaft along a second rotation axis substantially parallel to the first rotation axis, the first and second magnetic rollers being positioned with a gap therebetween,
wherein each magnet in the first and second sets is diametrically magnetized with a magnetic orientation substantially perpendicular to the first or second rotation axis.

2. The system of claim 1, wherein the magnetic orientations of the adjacent magnets in the first or second set are angularly displaced.

3. The system of claim 2, wherein the magnets in each set are angularly displaced with an angle of 180°/N, where N is the number of magnets of the first or second set.

4. The system of claim 2, wherein the magnets in each set are angularly displaced with unequal angles.

5. The system of claim 1, further comprising a pair of compensation magnets adjacent to the same ends of the first and second sets of magnets.

6. The system of claim 1, wherein the first set of magnets are arranged as first and second subsets side by side, and the second set of magnets are arranged as first and second subsets side by side, magnetically engaging with the first and second subsets of the first set of magnets, respectively.

7. The system of claim 6, wherein the magnetic orientations of the first and second subsets are angularly offset by about 90 degrees.

8. The system of claim 1, further comprising a mechanical system functionally connected to at least one of the first and second rotating shafts to convert between a magnetic potential energy and a mechanical potential energy of the system.

9. The system of claim 8, wherein the mechanical system comprises a cam fixed to at least one of the first and second rotating shafts, and a spring functionally connected to the cam via a cam roller.

10. The system of claim 1, further comprising a first motor mechanically connected to the first rotating shaft, and a second motor mechanically connected to the second rotating shaft.

11. The system of claim 1, wherein the gap between the first and second rollers are in a range from 0.01 cm to 50 cm.

12. The system of claim 1, wherein the first set of magnets and the second set of magnets are positioned such that a torque to rotate the rollers is substantially zero.

13. A method comprising:
positioning a first magnetic roller extending along a first rotation axis and a second magnetic roller extending along a second rotation axis substantially parallel to the first rotation axis, the first and second magnetic roller each including a first or second set of magnets mounted on a first or second rotating shaft, wherein each magnet in the first and second sets is diametrically magnetized with a magnetic orientation substantially perpendicular to the first or second rotation axis; and
rotating the first and second magnetic rollers with a torque.

14. The method of claim 13, further comprising reducing the torque to complete the rotation by angularly displacing the magnetic orientation of the adjacent magnets in each set.

15. The method of claim 14, wherein the magnets are angularly displaced with an angle of 180°/N, where N is the number of magnets of the first or second set.

16. The method of claim 14, wherein the magnetic orientations of the magnets in each set are angularly displaced with unequal angles.

17. The method of claim 13, further comprising reducing the torque to complete the rotation by disposing a pair of compensation magnets adjacent to the same ends of the first and second sets of magnets.

18. The method of claim 13, wherein the first set of magnets are arranged as first and second subsets side by side, and the second set of magnets are arranged as first and second subsets side by side, magnetically engaging with the first and second subsets of the first set of magnets, respectively.

19. The method of claim 18, wherein the magnetic orientations of the first and second subsets are angularly offset by about 90 degrees.

20. The method of claim 13, further comprising functionally connecting a mechanical system to at least one of the first and second rotating shafts to convert between a magnetic potential energy and a mechanical potential energy of the apparatus.

21-23. (canceled)
